(19) United States
(12) Patent Application Publication
(10) Pub. No.: US 2019/0269518 A1
(43) Pub. Date: Sep. 5, 2019

(54) CEMENTLESS JOINT RESURFACING SYSTEM

(71) Applicant: RAYCONT LTD., Yad Natan (IL)

(72) Inventor: Gad SHMUELI, Yad Natan (IL)

(21) Appl. No.: 16/320,185

(22) PCT Filed: Jul. 26, 2016
(86) PCT No.: PCT/IL2016/050818

§ 371 Date: Jan. 24, 2019

(51) Int. Cl. A61F 2/38; A61F 2/30

(52) U.S. Cl. CPC A61F 2/3859; A61F 2/3877; A61F 2/4202; A61F 2/30756; A61F 2/389

(57) ABSTRACT

A cementless individual patellofemoral resurfacing system including a femoral lamellar sheet having an articular surface and a bone covering surface, at least one rib attached to the femoral lamellar sheet along at least one long border thereof, a patella lamellar sheet having a patella articular surface and a patella covering surface non-breachingly attached to bone in a joint-sparing manner. The cementless patellofemoral resurfacing system can also serve as a central supporting component for at least one attachable and detachable condylar articular surface resurfacing elements. The system can be implemented mutatis mutandis in other bony joints in the body.

### FIELD OF TECHNOLOGY

The system relates to bony joint resurfacing and in particular to cementless patellofemoral resurfacing systems.

### BACKGROUND

In orthopedic procedures the need often arises to reconstruct damaged or worn out surfaces of cartilage covering the articular surfaces of the femur condyles, the patellar groove or patellar surface between the condyles and the articular surface of the patella in contact with and gliding along the patellar groove of the femur. Continued deterioration of the knee joint cartilage can eventually lead to a need for a total knee joint replacement (TKR).

Several orthopedic techniques have been developed in an attempt to enable fast as possible restoration of functionality of the knee joint loss of function due to patellofemoral cartilage degeneration or trauma. The goal of these techniques has also been to use procedures less extensive than TKR.

Some of the techniques, such as that disclosed in PCT Application No. WO9743985 were named “Replacement” techniques, disclosing a procedure which involves extensive preparation of the bone by removal of the damaged articular surfaces so that to accept an artificial replacement articular surface prostheses. These procedures fall only slightly short of a full TKR procedure.

Other techniques prevalent in the art are named “Resurfacing” techniques, which disclose resurfacing of the damaged articular surfaces. However many of the disclosed “Resurfacing” techniques, appear to be in most cases replacement techniques, replacing the natural cartilage-covered articular surface with an artificial articular surface in which the artificial articular surface mimics the natural articular surface. Though less aggressive than “Replacement” techniques, most disclosed procedures still involve some type of preparation of the site to be resurfaced or replaced by either superficial removal of bone tissue or drilling of holes into the bone tissue through the articular surface and cartilage to accept pins attached to the resurfacing prosthesis to be attached. These types of procedures are time consuming, lengthen the overall surgical time and time under anesthesia and require an extended healing and recuperation period and slow return to functionality.

Moreover, the current techniques involve some degree of alteration or removal of natural soft tissue joint components such as one or both cruciate ligaments. Overall, the natural knee joint is altered so that to accommodate the artificial implant transferring all of the functionality of the joint natural components to the implanted prostheses.

Many of the “resurfacing” techniques also disclose using bone cement (e.g., Poly(methyl methacrylate) or PMMA) to adhere the prosthesis to the bone by coating the prosthesis and/or bone surface with bone cement in an attempt to provide permanent or at least long term contact between the bone and/or prosthesis. In some cases this involves adding pins to the prostheses so that to increase the area of contact of bone cement between the prostheses and the bone. Bone cement attachment also requires total removal of existing cartilage. Two such examples are U.S. Pat. No. 6,905,514 and U.S. Pat. No. 4,353,135.

However, high temperatures of bone cement during application can damage surrounding healthy bone cells. Bone cement can also provoke a possible immune response. Additionally, not just does bone cement not promote osseous integration there is commonly wear of the bone cement leading to loosening of the prosthesis-bone contact surface. This occurs as a result of synovial fluid seeping into and along the contact surface between bone and prosthesis, loosening and replacing the bone cement. This phenomenon may limit the functional lifetime of the treated joint and may eventually lead to a need for replacement of the prosthesis. In such cemented systems, however, late revisions often require removal of all components including the bone cement. Alternatively, cementless attachment of a resurfacing apparatus especially when employing an anchor attachment system brings about a stronger bond that becomes stronger over time as a result of osseous integration. In such systems infection rate is very low as well. Late revision, in rare instances, do not require any interruption of the integrity of boney and/or cartilaginous structure of the joint and of bone cement and operation time is dramatically reduced shortening time under anesthesia and limiting blood loss.

### SUMMARY

The instant document discloses a cementless patellofemoral resurfacing system and method that can be mutatis mutandis be implemented in any bony joint in the body.

The system can be used for resurfacing bony joints such as, for example, worn patella and adjacent femoral joint cartilage resulting from trauma or mal alignment as part of the PFPS (Patello-Femoral Pain syndrome). The system can replace Total Knee Replacement (TKR) procedures that replace the whole knee and are usually adhered to the exposed spongy bone with polymethylmethacrylate (PMMA) as cement.

The system is based on a patient's MRI findings which make it pre-adapted to a specific individual joint. It consists of preserving bone and cartilage by coverage of the damaged (worn) cartilage and providing when needed a tracking canal that prevents subluxation of the joint or, e.g., patella, and offers a better contact between the articulating parts.

Measurement and fitting of the implant as mentioned previously are prepared on data extracted from a MRI image of the patient's knee so that upon insertion at operation theater the form fits exclusively to this certain patient. All parts of the present solution could be produced by a 3D printing system. A set of tools is provided with the implant, so that there is no need to keep a stock of implants and probes, or to prepare a large number of instruments.

The solution that the present document offers can be inserted and fixed without any changes to joint cartilage, and cruciate ligaments are preserved.

This procedure is done by minor surgery with a fast recovery and less suffering. The most important feature of the procedure is that it is reversible. This means that in unsuccessful cases, the devices can be removed, contrary to the existing solution which uses resection of bone and cementing or by performing a full TKR (Total Knee Replacement) in case of failure. A TKR-failure ends with arthrodesis and shortening of the leg.

The cementless patellofemoral resurfacing system and method includes a femoral lamellar sheet having an articular surface and a bone covering surface. The material from which the lamellar sheet is made and the thickness thereof are designed to protect underlying tissue (cartilage and/or bone) and are set at a minimum only sufficient to withstand forces of friction and pressure applied thereto by a patella gliding over and along a trough in the articular surface of the lamellar sheet.

In one example the system also includes a patella lamellar sheet formed to have a protrusion-like cross section forming a patella articular surface that generally follows and parallels the femoral articular surface of the femoral lamellar sheet.

In other examples the cross-sections of the articular surfaces of the femoral lamellar sheet and patella lamellar sheet can have any suitable geometry as long as they parallel each other so that to allow one to glide along the other without any hindrance.

In still another example the patellofemoral resurfacing system can be expanded to form a cementless arthroplasty resurfacing system wherein the femoral lamellar sheet can serve as a central supporting component for additional structural components of a full arthroplasty procedure such as condylar resurfacing elements or condylar replacement prostheses as well as tibial articular resurfacing components can also be added.

In yet another example the patellofemoral resurfacing system can include one or more slots along which the patellofemoral resurfacing system can glidingly accommodate one or more ridges attached to a portion of an edge of condylar articular surface resurfacing elements so that together all surfaces complete a full arthroplasty resurfacing and protective layer.

The patellofemoral resurfacing system femoral lamellar sheet serving as a central supporting component also obviates some attachment tabs lessening the required amount of drilling in the bone and shortening the length of the procedure.

In still another example patellofemoral resurfacing system can also be designed to accommodate a universal implant-to-bone fixation system.

In still another example the patellofemoral resurfacing system can be individually fitted by 3-D titanium printing technology.

In another example, the patellofemoral resurfacing system and/or cementless arthroplasty resurfacing system are attached to bone/cartilage cementlessly and non-breachingly and in a joint-sparing manner employing a sawless procedure.

In still another example, the cementless arthroplasty resurfacing system can also include a change to the femoral outer surface so that to include a stopper that when articulating together with a flat articular surface of a tibial articular resurfacing components can prevent any undesired hyper-extension of the knee joint.

The manufacturing of a cementless patellofemoral resurfacing system by 3-D titanium printing technology supports individual fitting of the system to each patient individually as well as the addition of features as required by the individual patient such as, for example, a hyper-extension prevention system.

### BRIEF DESCRIPTION OF THE SEVERAL VIEWS OF THE DRAWINGS

FIGS. 1A, 1B and 1C are perspective view simplified illustrations of patellofemoral resurfacing systems in accordance with three examples;

FIGS. 2A and 2B are cross-section view simplified illustrations of the cementless patellofemoral resurfacing system of FIG. 1 with a patella placed at the same section level in accordance with an example;

FIG. 3 is a perspective view simplified illustration of implementation of the cementless patellofemoral resurfacing system of FIGS. 1A-C and 2A-B depicting the spatial relationship between the patella and the trough or groove of the cementless patellofemoral resurfacing system of FIG. 1;

FIGS. 4A, 4B, 4C and 4D are cross-section view simplified illustrations of cementless patellofemoral resurfacing systems in accordance with four examples;

FIGS. 5A and 5B are perspective view simplified illustrations of a cementless patellofemoral resurfacing system with an attached pair of condylar articular surface resurfacing elements in accordance with yet another example;

FIGS. 6A, 6B and 6C are cross-section and perspective view simplified illustrations of a hyper-extension prevention system of a cementless patellofemoral resurfacing system to bone in accordance with another example;

FIG. 7A, 7B, 7C, 7D and 7E are perspective view and cross-section view simplified illustrations of a cementless patellofemoral resurfacing system to which a pair of condylar articular surface resurfacing elements are attached in accordance with another example;

FIG. 8A, 8B, 8C, 8D and 8E are perspective view and cross-section view simplified illustrations of a cementless patellofemoral resurfacing system to which a pair of condylar articular surface resurfacing elements are attached in accordance with still another example;

FIGS. 9A, 9B and 9C are cross-section and perspective view simplified illustrations of an attachment method of a cementless patellofemoral resurfacing system to bone in accordance with another example; and

FIGS. 10A, 10B and 10C, which are perspective view simplified illustrations of a tibiotalar resurfacing system in accordance with an example.

### DETAILED DESCRIPTION

The term “Lamellar Sheet” as used in this disclosure means a thin sheet-like structure that is attached to an articular surface of a bone in a non-breaching manner.

The terms “Non-Breaching” and “Non-Breachingly” as used in this disclosure means an attachment and method of attachment to bone of a structure such as, for example, a lamellar sheet, without any disruption to, or alteration of the integrity of the existing articular surface 302 (FIG. 3) of the bone and/or cartilage to which the attachment is to be made regardless of their condition.

The term “Joint Sparing” as used in this disclosure means a surgical procedure of the knee in which none of the soft tissue components of the knee joint inside and outside the capsule, such as ligaments, are removed, altered or damaged.

The term “Para-Cartilage” as used in this disclosure means non intra-articular, non-cartilage covered areas of the bone.

One advantage of the disclosed system and method is in that it is designed to provide a joint sparing non-breaching system for repair of knee joint damage and loss of functionality in a manner that requires a short and relatively simple surgical procedure and enables a relatively rapid recovery allowing almost immediate return to functionality. Another advantage of such a non-breaching procedure is in that should replacement of the system later in time be required for any reason or a different procedure is later elected to be performed, the disclosed system can be removed without any anatomical change of the underlying bone and cartilage allowing for an unlimited choice of any type of elected orthopedic procedure to be performed.

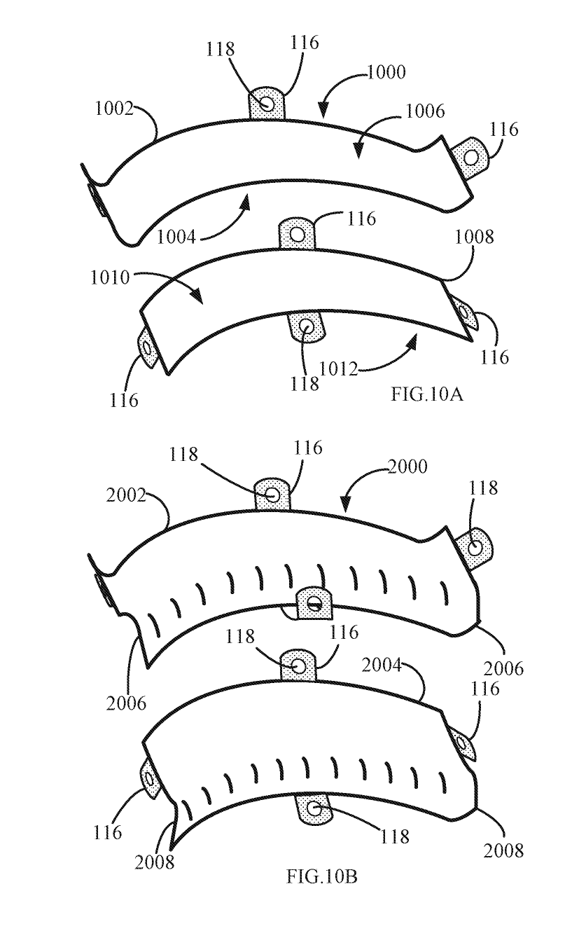

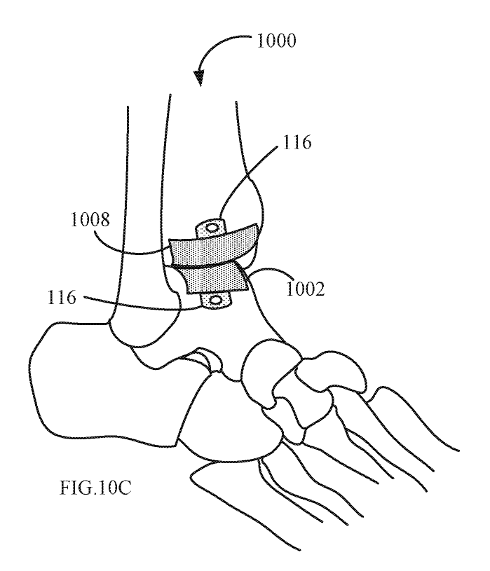

Although the following text brings forth primarily examples of implementation of the disclosed system relating to the knee, being one of the more complex and frequently damaged bony joints in the body, it will be appreciated by persons skilled in the art that the disclosed system can be implemented mutatis mutandis in other joints in the body such as, for example, the elbow joint or ankle joint as illustrated in the example brought forth in FIGS. 10A, 10B and 10C.

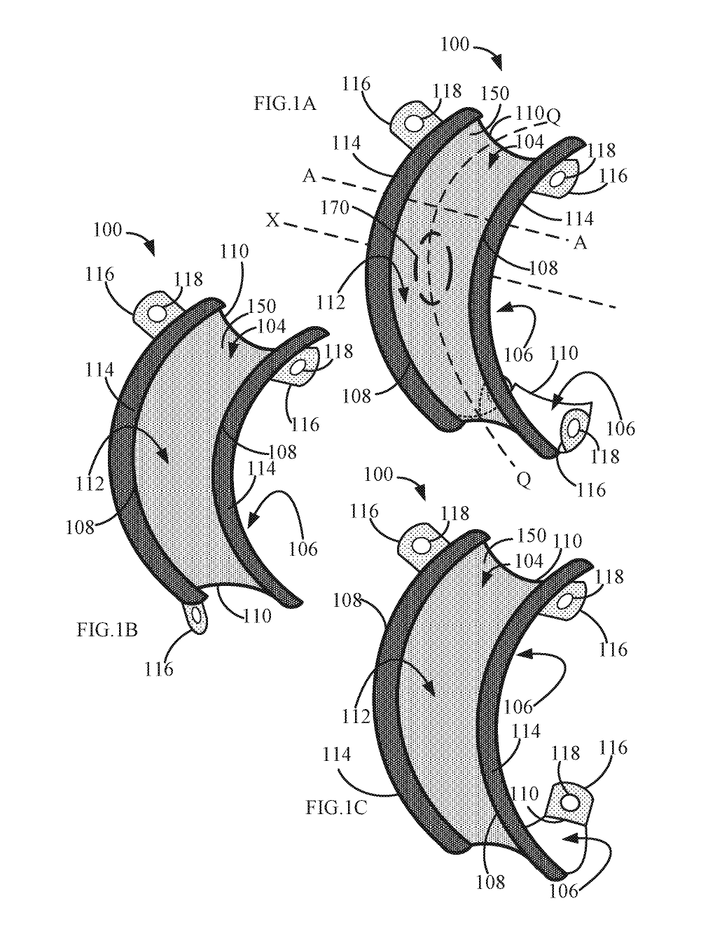

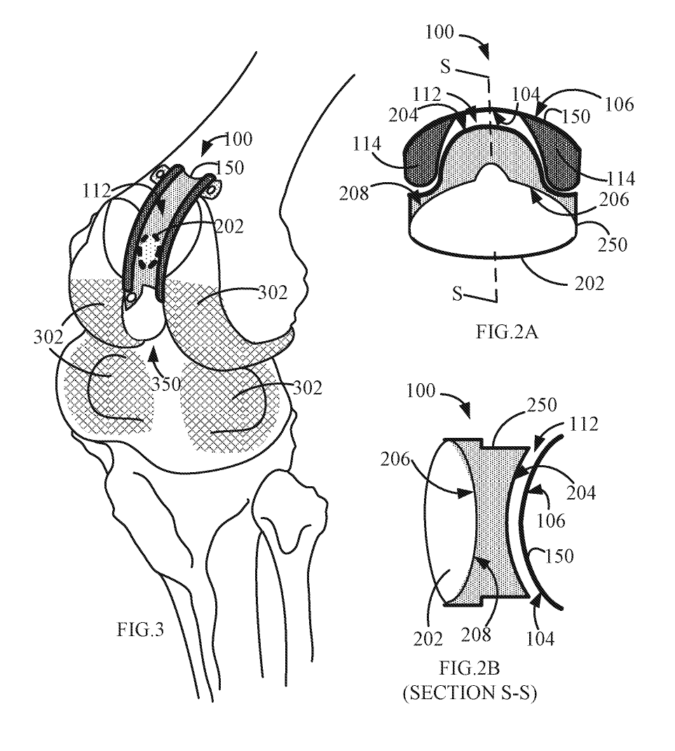

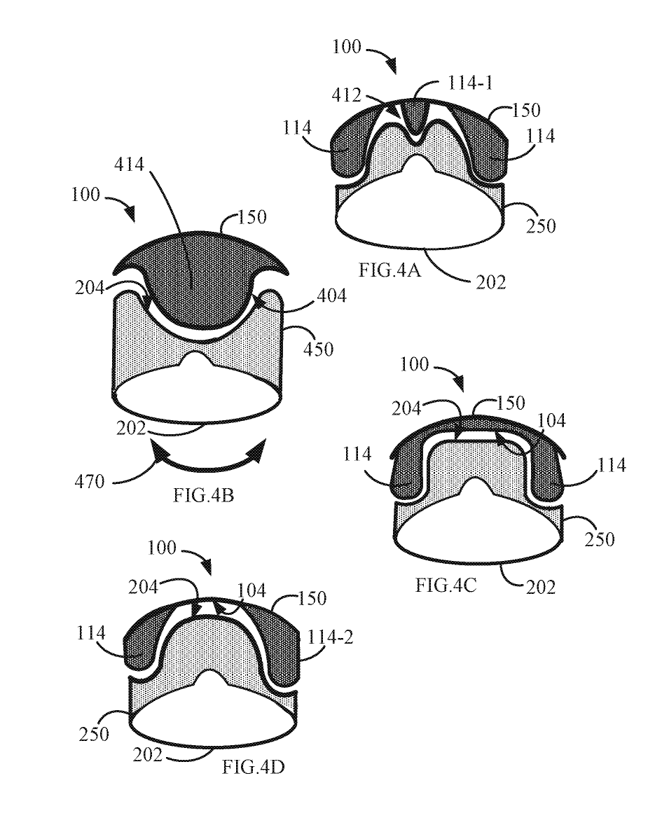

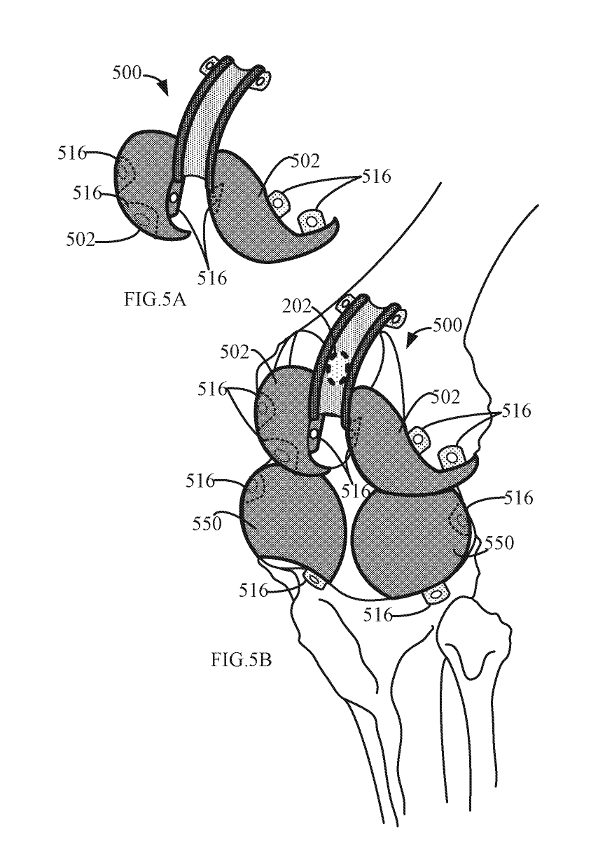

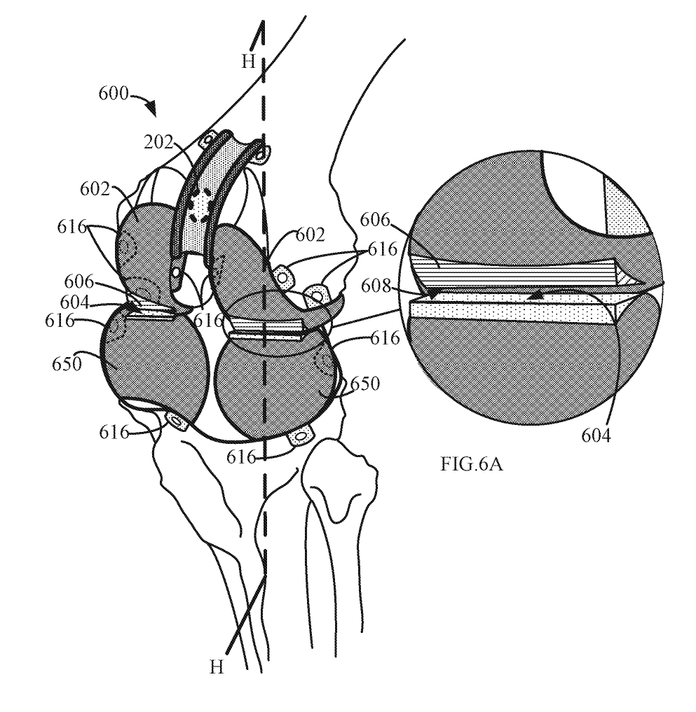

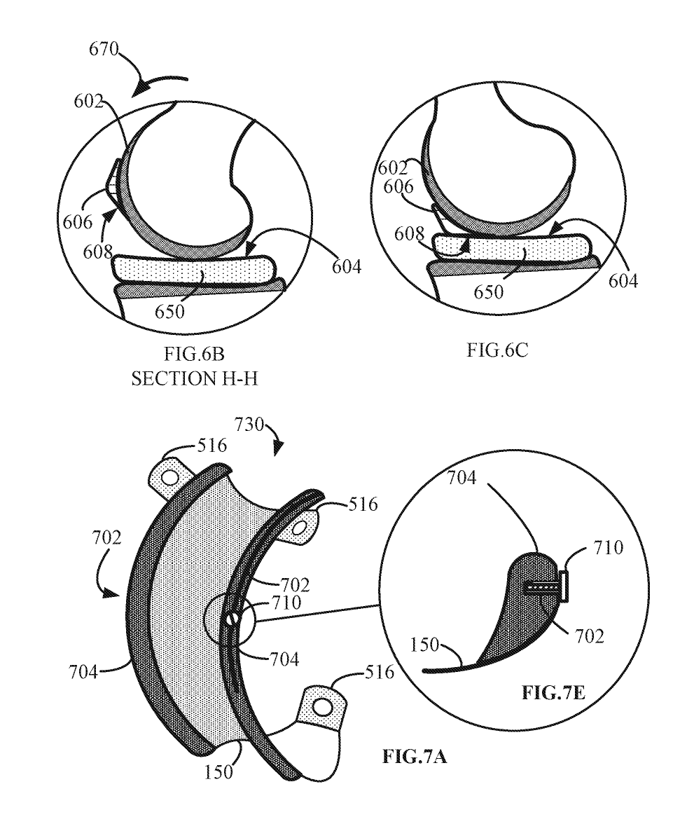

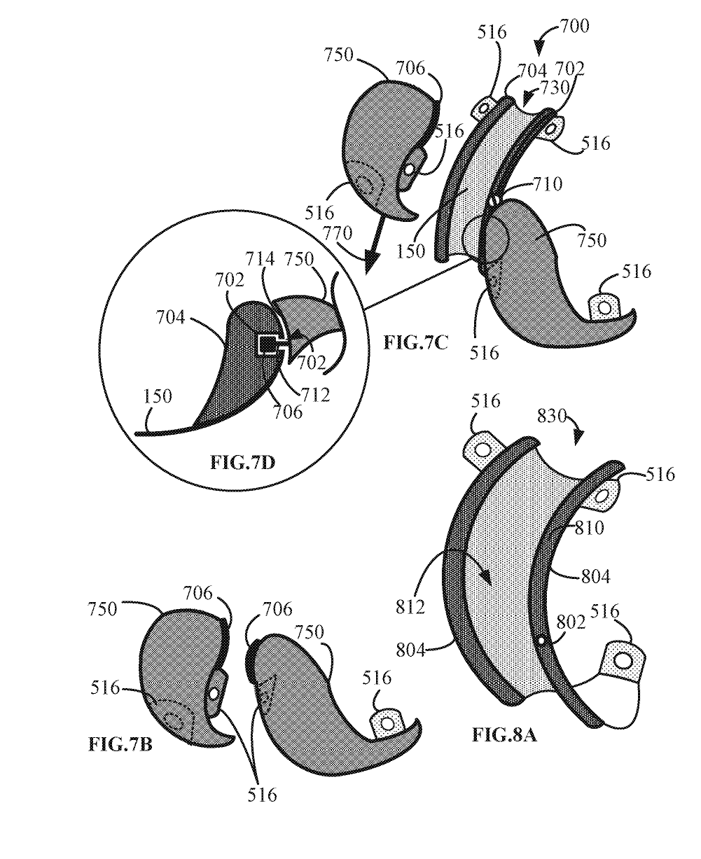

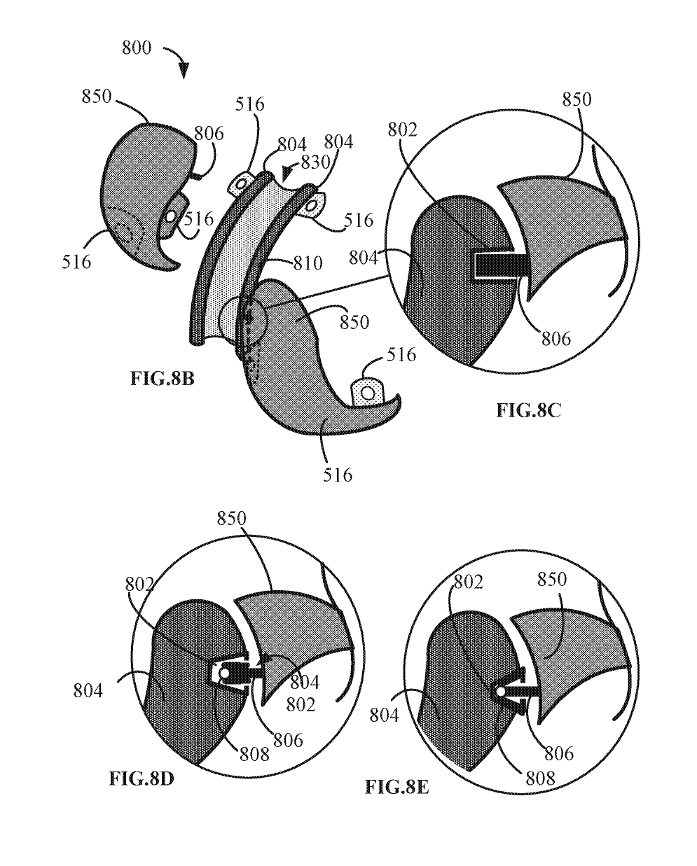

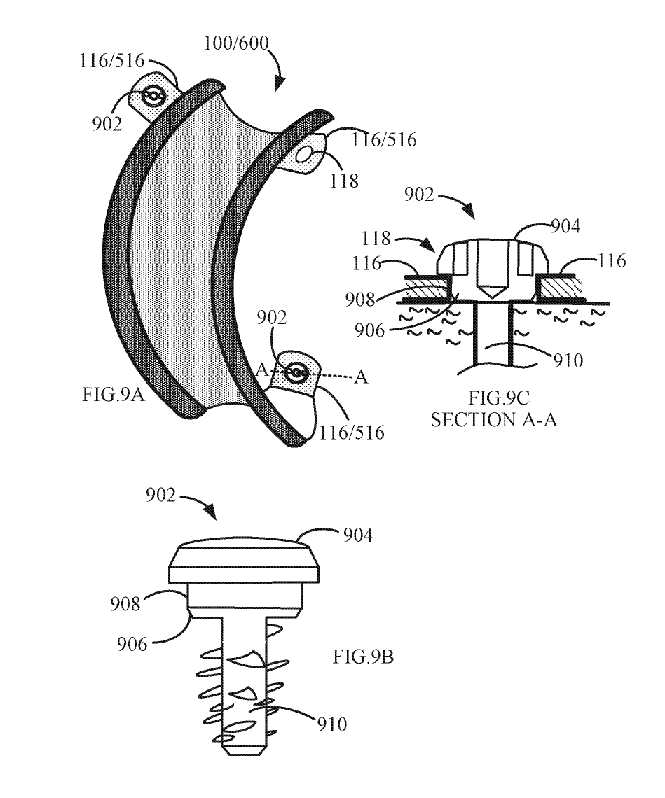

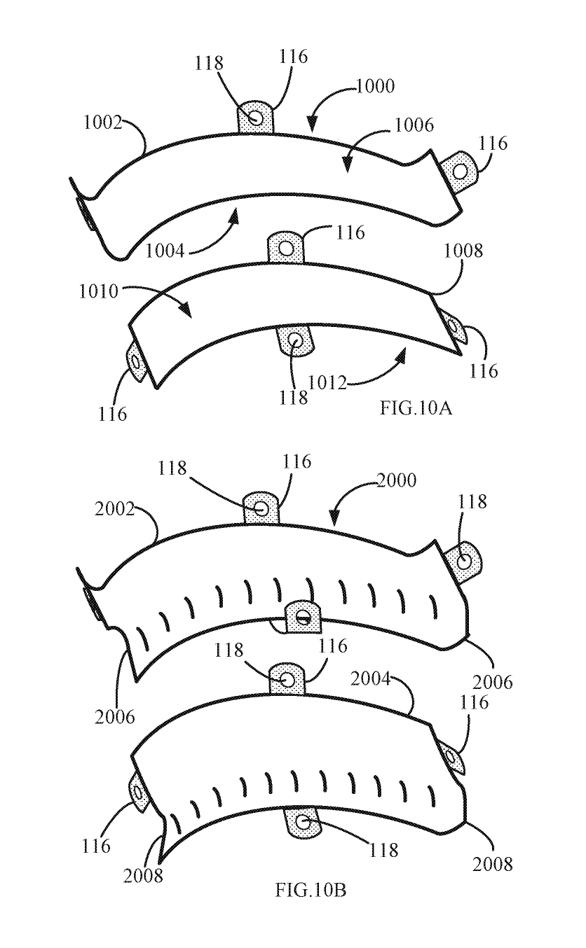

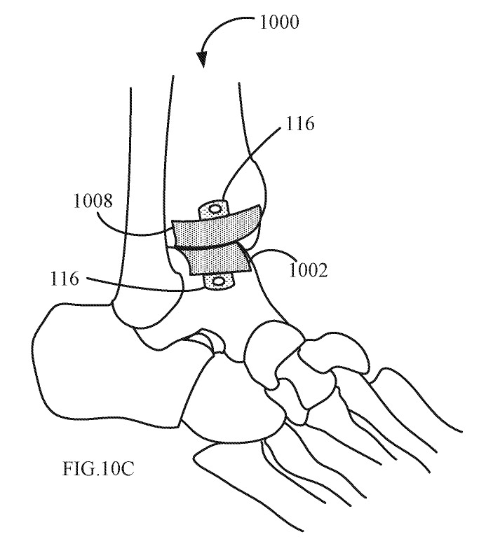

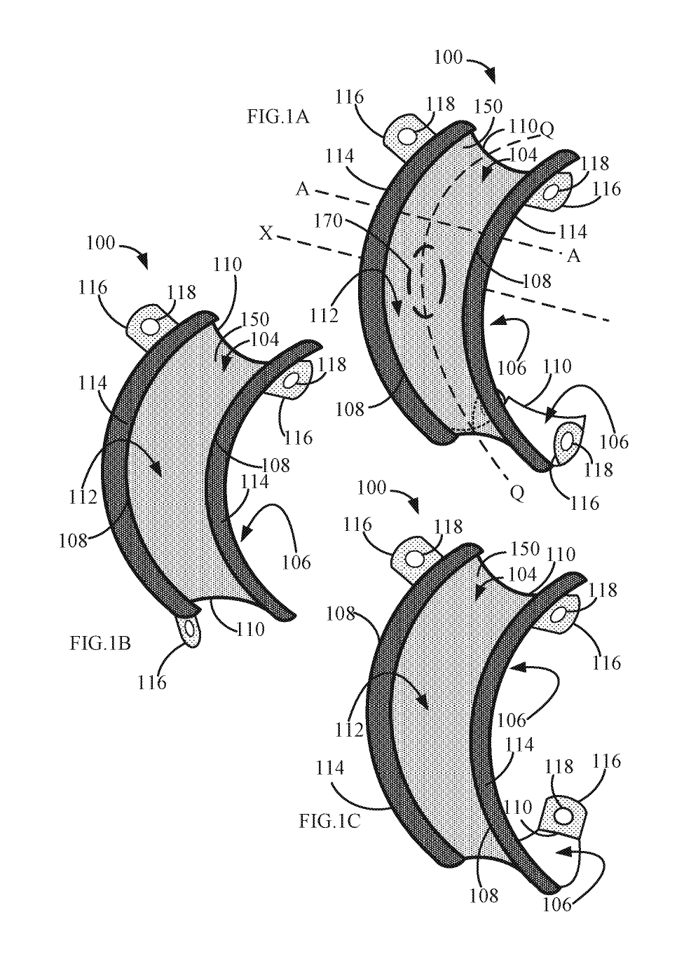

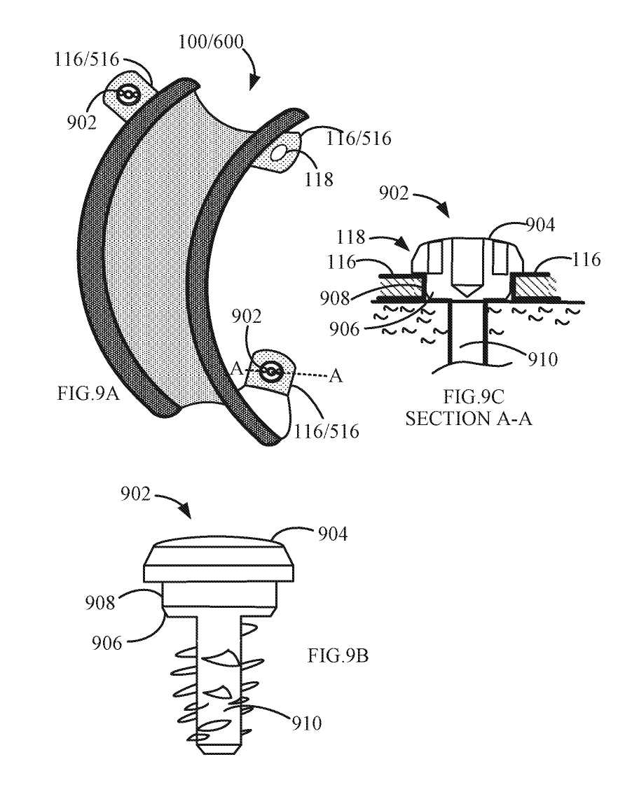

Reference is made to FIGS. 1A, 1B and 1C, collectively referred to as FIG. 1, which are perspective view simplified illustrations of a cementless joint resurfacing system in accordance with three examples. System 100, is a cementless patellofemoral resurfacing system that can include a femoral lamellar sheet 150 having an articular surface 104 and a bone and/or cartilage (B/C) covering surface 106. B/C covering surface 106 can non-breachingly cover healthy or damaged bare bone, cartilage covered bone or partially cartilage covered bone regardless of their condition. Attachment of femoral lamellar sheet 150 to bone can involve a sawless procedure requiring only a ratchet-type instrument to apply anchors 902 (FIG. 9) through one or more tabs 116 one or more anchor eyes 118 into para-cartilage bone.

Femoral lamellar sheet 150 can be generally curved on at least a portion of a radius about an imaginary axis X (FIG. 1A) and be bound by long borders 108, and also curved on at least a portion of a radius about a femoral lamellar sheet 150 longitudinal axis Q-Q (FIG. 1A) bound by short borders 110 so that to form a trough or groove 112 along axis Q-Q bound by long borders 108.

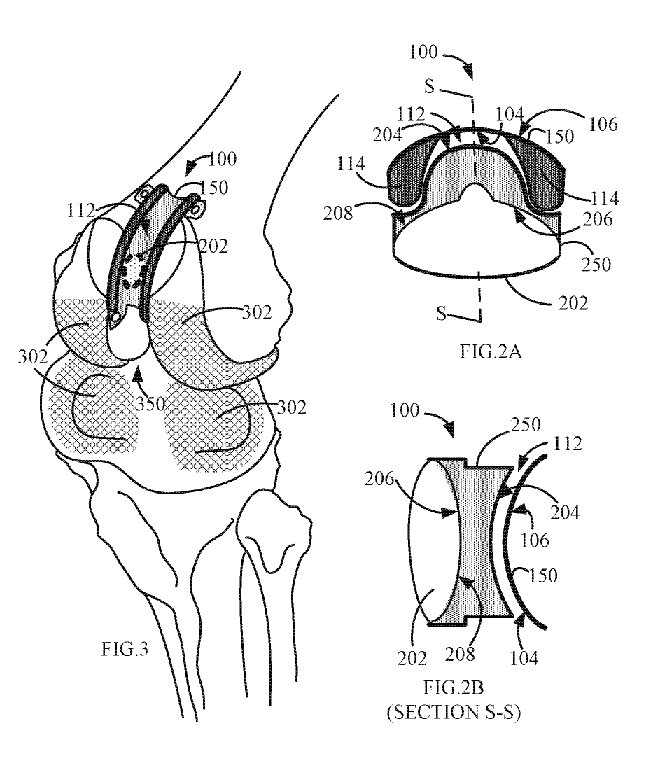

Femoral lamellar sheet 150 can function as a protective cover, a bandage of sorts, protecting damaged underlying bone or cartilage from further erosion as well as provide an articular surface over and along which a patella 202 (FIG. 2) can glide. Hence, femoral lamellar sheet 150 is not embedded in bone and therefore can protrude above the surrounding surface of bone and/or cartilage. The added height resulting from the protrusion of femoral lamellar sheet 150 above the surrounding surface of bone and/or cartilage can be corrected for by adjustment of femoral lamellar sheet 150 articular surface and a patella 202 articular surface 204 (FIG. 2).

The material from which the lamellar sheet is made and the thickness thereof can be designed to provide a protective cover for underlying tissue (cartilage and/or bone) having a minimal or no negative affect thereupon and have a minimal thickness only sufficient to withstand forces of friction and pressure applied thereto by patella 202 (FIG. 2) gliding over and along trough 112 in the articular surface of femoral lamellar sheet 150.

The thickness of femoral lamellar sheet 150 can be uniform throughout or variable. However, the forces (friction and/or pressure) applied to articular surface 106 of femoral lamellar sheet 150 vary at various points on surface 106. For example, forces at the upper parts, farthest from a center portion 170 of articular surface 106 are minimal and mainly protect from patellar luxation or subluxation. Further down groove 112 and closer to center portion 170 the forces tend to increase. Therefore, in one example, the thickness of femoral lamellar sheet 150 can vary in a gradient being maximal in a center portion 170, gradually decreasing in thickness towards borders 110/108 of femoral lamellar sheet 150.

One or more ribs 114 can be attached to femoral lamellar sheet 150 along long borders 108 and sandwich trough or groove 112 therebetween so that to form guard-rail like structures that limit excessive lateral movement (i.e., luxation or sub-luxation) of patella 202 gliding over femoral lamellar sheet 150. The height of ribs 114 from articular surface 106 along the length of ribs 114 can be uniform or variable as will be explained in greater detail below. Two or more ribs 114 on either side of trough or groove 112 can be equal in height or differ in height from each other.

Attachment tabs 116 including anchor eyes 118 can be attached to borders 108/110 of femoral lamellar sheet 150 at various locations so that to accommodate attachment anchors 902 (FIG. 9) and be located over para-cartilage areas of the joint so that to attach cementless patellofemoral resurfacing system 100 to bone in a joint sparing manner as will be described in greater detail below.

Femoral lamellar sheet 150 can be made of a biocompatible material such as titanium or a titanium alloy and have a thickness of between 1 mm and 3 mm, more commonly between 1.5 mm and 2.5 mm and most commonly 2 mm. Femoral lamellar sheet 150 can be manufactured with 3-D titanium printing technology so that to enable precise individually fitting of femoral lamellar sheet 150 to the boney and/or cartilaginous surface to be covered. 3-D printing can also enable variations in the morphology of trough or groove 112 as desired (e.g., to correct for protrusion of protrusion of femoral lamellar sheet 150 above the surrounding surface of bone and/or cartilage as described above) and to control specific directional movement of the patella along trough or groove 112 as will be demonstrated in greater detail below.

Additionally and optionally, B/C covering surface 106 can be fully or partially coated with a micro-granular titanium or titanium alloy layer of micro-trabeculae such as, for example, Trabecular Structures™ (Arcam AB® Krokslatts Fabriker, 27A, SE-431 37 Mölndal, Sweden) to further stimulate bone and/or cartilage growth into micro trabeculae increasing surface contact strength with underlying tissue and limiting its movement after anchoring. Such a micro-granular layer renders femoral lamellar sheet 150 not only non-breaching but also a bone/cartilage growth-promoting apparatus.

FIGS. 2A and 2B, together referred to as FIG. 2 are cross-section view simplified illustrations of cementless patellofemoral resurfacing system 100 at the level of section A-A (FIG. 1A) together with a patella 202 placed at the same section level A-A in accordance with an example. FIG. 2B is a cross-section view of the system 100 of FIG. 2A at the level of section S-S of FIG. 2A. Patella 202 can be resurfaced by a patella lamellar sheet 250 having an articular surface 204 and a patella covering surface 206. Attachment tabs 116 including anchor eyes 118 can be attached to borders 208 of lamellar sheet 250 at various locations as desired to be located over para-cartilage areas of the bone so that to attach cementless patella lamellar sheet 250 to the patella in a joint sparing manner.

Patella lamellar sheet 250 can be made of a biocompatible polymer material and have a thickness of less than 3 mm, commonly between 1 mm and 3 mm, more commonly between 0.5 mm and 2.0 mm and most commonly 1 mm. Patella lamellar sheet 250 can be manufactured with 3-D printing technology so that to support precise individually fitting of Patella lamellar sheet 250 to the boney surface of patella 202 to be covered. 3-D printing can also enable variations in the morphology of patella articular surface 204 as desired to control specific directional movement of patella 202 along trough or groove 112 and height variations of femoral lamellar sheet 150 above surface of surrounding bone and/or cartilage as will be demonstrated in greater detail below.

Patella lamellar sheet 250 can be formed to have a protrusion-like cross section so that patella articular surface 204 generally follows and parallels femoral articular surface 104 of femoral lamellar sheet 150. This enables gliding of patella lamellar sheet 250 articular surface 204 over femoral lamellar sheet 150 articular surface 104 when flexing and extending the knee joint.

Patella lamellar sheet 250 can cover an articular surface 208 of patella 202 and in some cases can cover most of the surface, i.e., beyond the articular surface 208, of patella 202.

FIG. 3 is a perspective view simplified illustration of implementation of cementless patellofemoral resurfacing system 100 depicting the spatial relationship between system 100 and articular surfaces 302 as well as between patella 202 and trough or groove 112. As shown in FIG. 3 and described above, since no alteration is performed of the bone or cartilage surface underlying femoral lamellar sheet 150, after attachment, femoral lamellar sheet 150 can protrude above the surrounding surface of bone and/or cartilage.

As shown in FIG. 3, system 100 is designed as a joint sparing system so that no component thereof (i.e., femoral and patellar lamellar sheets 150/250, attachment tabs 116/516 and ribs 114) interferes with the integrity and function of non-damaged components of the knee joint. Tabs 116 are also located in joint sparing locations as well such as over para-cartilage surfaces of the bone.

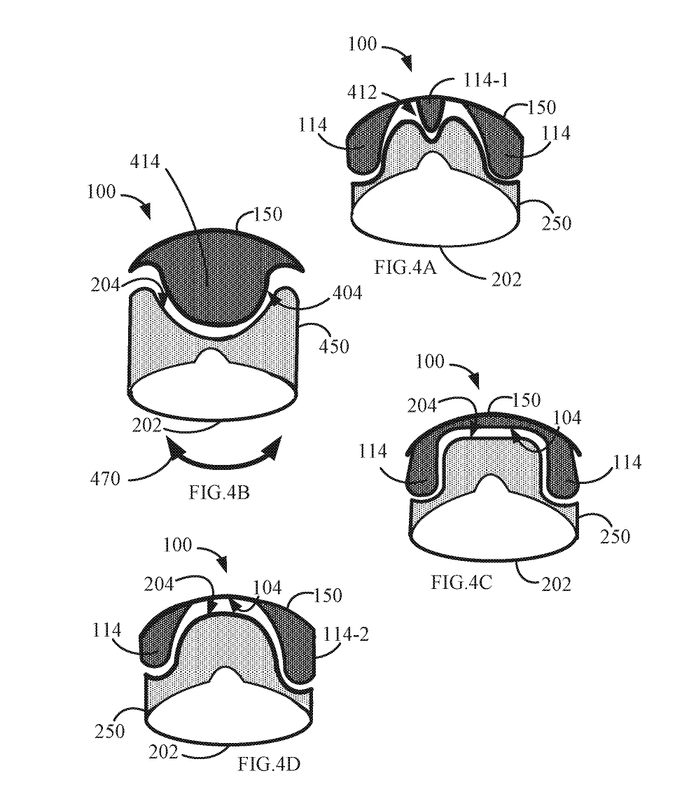

Referring now to FIGS. 4A, 4B, 4C and 4D collectively referred to as FIG. 4, which are cross-section view simplified illustrations of examples of system cementless patellofemoral resurfacing system 100. Articular surfaces 104 and 204 do not necessarily need to mimic the natural articular surface of the bones of the knee joint and they can be designed and fitted for specific orthopedic situations that require unconventional and unique solutions or to correct for protrusion of femoral lamellar sheet 150 above the surrounding surface of bone and/or cartilage as described above. Employing 3-D printing technology can increase the precision of fit of each uniquely manufactured system 100 articular surfaces 104/204 for the individual need of each and every case.

As shown in FIG. 4, femoral lamellar sheet 150 and patella lamellar sheet 250 do not necessarily need to be designed to include a single trough (femoral lamellar sheet 150) and single protrusion-like (patella lamellar sheet 250) cross-section as shown, for example, in FIG. 2. As depicted in FIG. 4A, for example, femoral lamellar sheet 150 can include an additional rib 114-1 along the center (“deepest” portion) of trough or groove 112 (FIG. 2) so that to form a trough or groove 412 having a W-shaped cross-section.

When compared to the example of FIG. 2, the example of FIG. 4A limits further any lateral deviation (luxation or sub-luxation) of patella 202 as it glides along trough or groove 412.

FIG. 4B depicts another example in which femoral lamellar sheet 150 can include a single rail-like rib 414 having a single protrusion-like cross-section along the center (“deepest” portion) of trough or groove 112 (FIG. 2). In this configuration, rib 414 includes an articular surface 404. Patella lamellar sheet 450 can include a single trough-shaped patella articular surface 204 that rides over rail-like single rib 414 articular surface 404. The example of FIG. 4B allows patella 202 more freedom of lateral angular deviation in an arc-form manner when patella 202 rides over rail-like single rib 414 as indicated in FIG. 4B by an arrow designated reference numeral 470.

FIG. 4C illustrates femoral lamellar sheet 150 articular surface 104 and patella lamellar sheet 250 articular surface 204 having generally parallel square cross-sections. It will be appreciated by a person skilled in the art that the articular surfaces of femoral lamellar sheet 150 and patella lamellar sheet 250 can have any suitable geometry as long as they parallel each other so that to allow one to glide along the other without any hindrance such as, for example, excessive friction.

FIG. 4D depicts cementless patellofemoral resurfacing system 100 designed for cases of asymmetrical patellar deviation such as, for example, patellar luxation or sub-luxation (“unstable kneecap”). In this example, a single rib 114-2 of a pair or more ribs 114 of femoral lamellar sheet 150 protrudes more from articular surface 104 of femoral lamellar sheet 150 surface than its paired rib on the opposite side of femoral lamellar sheet 150. Patellar lamellar sheet 250 articular surface 204 can be designed to parallel femoral lamellar sheet 150 articular surface 104 so that any tendency of patella 202 to luxate or sub-luxate in the direction of rib 114-2 while gliding over articular surface 104 of femoral lamellar sheet 150 is guarded and limited by rib 114-2. The determination of the rib to rise higher than its counterpart depends on the direction of the luxation or sub-luxation to be treated.

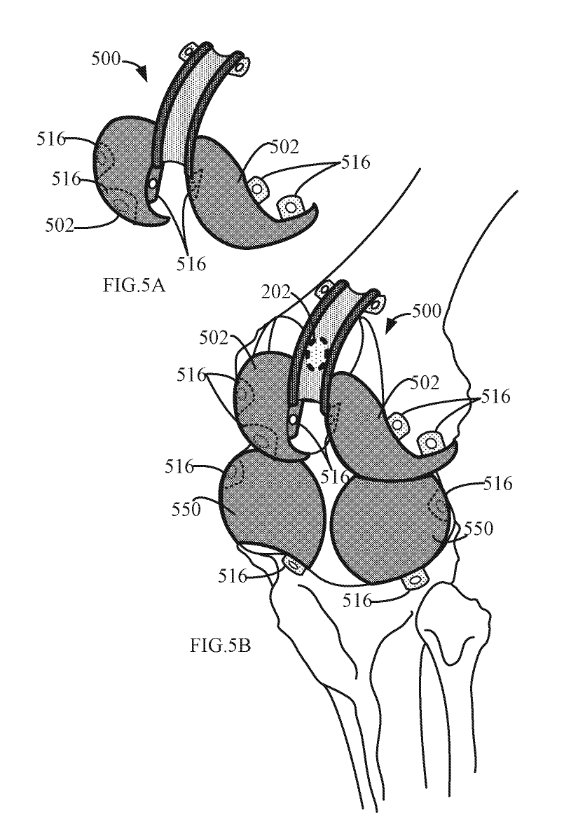

Reference is now made to FIGS. 5A and 5B collectively referred to as FIG. 5 and which are perspective view simplified illustrations of cementless patellofemoral resurfacing system 100 as a central component in total knee arthroplasty in accordance with an example.

In arthroplasty, it is very important for the implant to remain firmly attached to the underlying bone over time. Fixation of such implants to bone have originally employed bone cement. High temperatures of bone cement during application can damage surrounding healthy bone cells. Bone cement can also provoke an immune response and loosen over time (they really loose over time). Wear of the prosthesis-bone contact zone and may require replacement of the prosthesis however, late revisions often require removal of all components and bone cement.

Cementless attachment of a resurfacing apparatus brings about a stronger bond over a long term and infection rate is very low. Late revision do not require any interruption of the integrity of boney and/or cartilaginous structure of the joint and of bone cement and operation time is dramatically reduced shortening time under anesthesia. Less blood loss and shorter post-op treatment and follow up.

Most arthroplasty techniques in the art involve either “Replacement” techniques or less aggressive “Resurfacing” techniques that, as disclosed above, appear to be in most cases replacement techniques, replacing the natural cartilage-covered articular surface with an embedded-in-bone artificial articular surface prosthesis. Though less aggressive than “Replacement” techniques, most disclosed procedures still involve some type of preparation of the site to be resurfaced or replaced by either superficial removal of bone-cartilage tissue to accommodate an embedded prosthesis (or) and drilling of holes into the bone tissue through the articular surface and cartilage to accept pins attached to the resurfacing prosthesis to be attached by using of cement.

The advantage of cementless individually fitted patellofemoral resurfacing system 100 is in that that it is a non-breaching cementless attachment to bone system and can also be modularly expanded into, and serve as a central supporting component for, an arthroplasty resurfacing system including additional structural components of a full arthroplasty procedure. Other arthroplasty structural components can include condylar resurfacing elements or condylar replacement prostheses that can be attached on one or both sides of cementless patellofemoral resurfacing system 100.

FIGS. 5A and 5B illustrate a cementless expanded arthroplasty resurfacing system 500 in accordance with an example. One or more condylar articular surface resurfacing elements 502 can be attached to a cementless patellofemoral resurfacing system 100 together forming a cementless arthroplasty resurfacing system 500. The attachment of condylar articular surface resurfacing elements 502 can be single condylar or bicondylar femoral resurfacing attachment.

Condylar articular surface resurfacing elements 502 attached to and on either side of cementless patellofemoral resurfacing system 100 can include attachment tabs 516 for attachment of condylar articular surface resurfacing elements 502 to a para-cartilage surface of the bone. However, cementless patellofemoral resurfacing system 100 being a central supporting component for condylar articular surface resurfacing elements 502 and being itself firmly and tightly attached to the bone obviates the need for one or more attachment tabs 516 allowing for less drilling into bone and shortening of the length of time required for the arthroplasty procedure.

Cementless arthroplasty resurfacing system 500 can also include one or more tibial articular resurfacing components 550 to complement cementless arthroplasty resurfacing system 500. Tibial articular resurfacing components 550 can also include attachment tabs 516 for attachment thereof to a para-cartilage surface of the tibia.

Tibial articular resurfacing components 550 can be made of a biocompatible polymer and have a uniform or variable thickness as desired. In one example, one of components 550 can be thicker than the other to compensate for irregular or uneven gap size between femoral and tibial articular surfaces. In another example, one of components 550 can be thicker than the other to correct for, for example, Varus, Valgus or any other deformity in the knee joint.

FIG. 5B depicts an end result of a full arthroplasty procedure involving implementation of cementless arthroplasty resurfacing system 500 including cementless patellofemoral resurfacing system 100 serving as a central supporting component for condylar articular surface resurfacing elements 502, tibial articular resurfacing components 550 and patella 202. It will be appreciated that a full arthroplasty procedure employing the described resurfacing systems and components and in a manner described above renders the arthroplasty procedure a joint sparing, non-breaching, bone and/or cartilage growth promoting procedure.

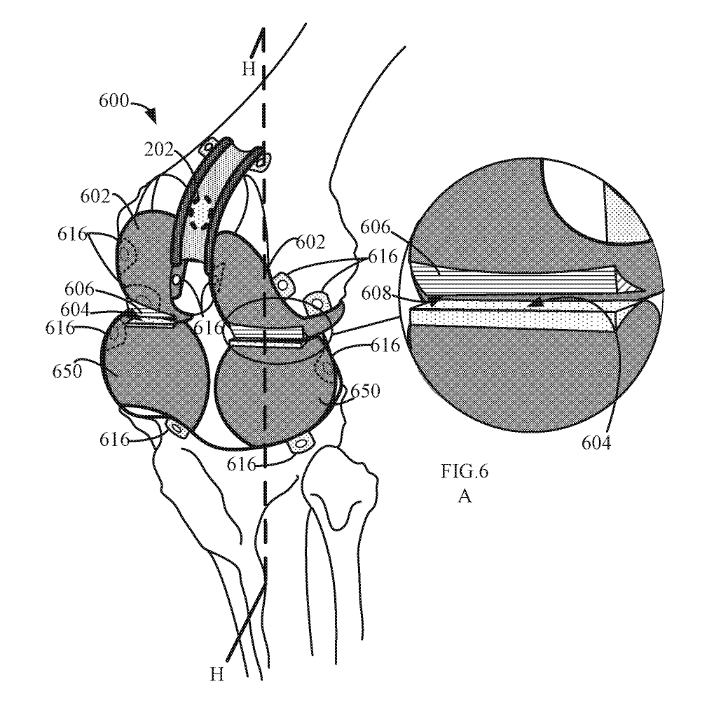

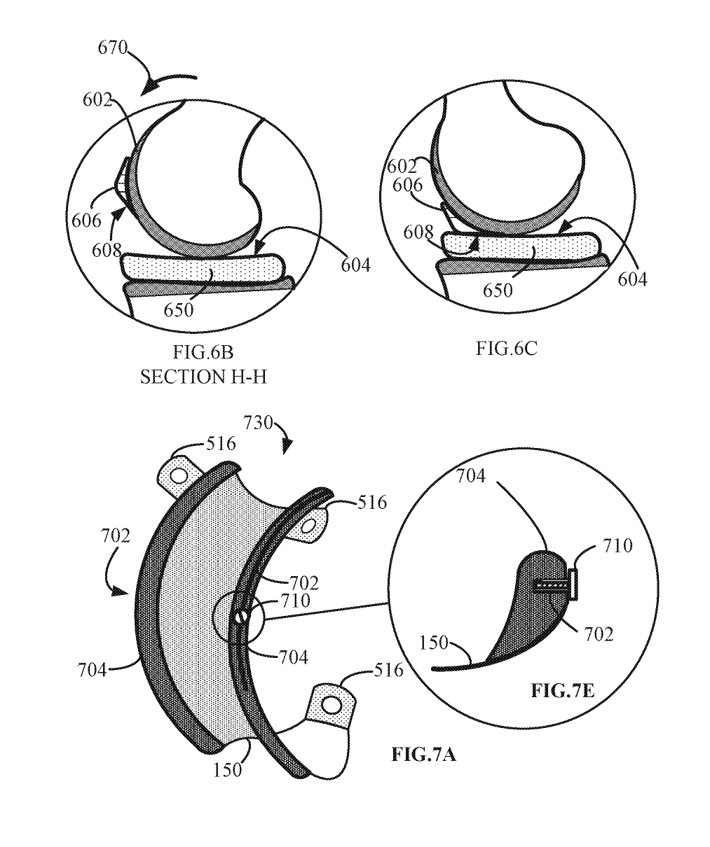

In another example, depicted in FIGS. 6A, 6B and 6C, together referred to as FIG. 6, a cementless arthroplasty resurfacing system 600 can include components 650 that can be designed to prevent recurvatum deformity such as occurs in cases of hyper-laxity in post-polio patients. Currently, such patients are treated only by a full Total Knee Replacement (TKR) procedure. Cementless arthroplasty resurfacing system 600 can thus include a change to the femoral outer surface so that to include a stopper that when articulating together with a flat articular surface 604 of tibial articular resurfacing components 650 can prevent any undesired hyper-extension of the knee joint. As illustrated in FIG. 6, at least a portion of one or more tibial articular resurfacing components 650 articulatingly abutting at least a portion of condylar articular surface resurfacing elements 602 can include a flat articular surface 604 supporting articular movement of condylar articular surface resurfacing elements 602 thereupon.

Condylar articular surface resurfacing elements 602 can include one or more hyper-extension stoppers 606 positioned on condylar articular surface resurfacing elements 602 so that to support fully functional articulation of the femur and tibia knee joint while concurrently, when the femur is rotated in a direction indicated in FIG. 6B by an arrow designated reference numeral 670, a contact surface 608 of one or more hyper-extension stoppers 606 can be urged against tibial articular resurfacing components 650 flat articular surface 604 as shown in FIG. 6C and thus can prevent undesired hyper-extension of the knee joint.

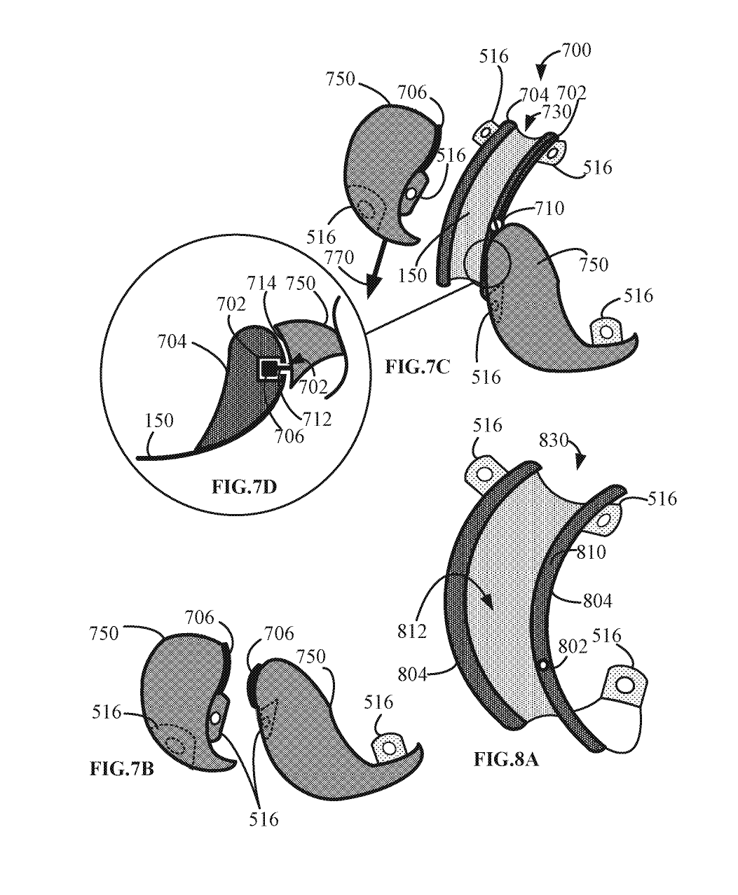

Reference is now made to FIGS. 7A, 7B, 7C, 7D and 7E, together referred to as FIG. 7, which are perspective view and cross-section view simplified illustrations of a cementless arthroplasty resurfacing system 700 in accordance with yet another example, which includes cementless patellofemoral resurfacing system 730 and an attachable pair of condylar articular surface resurfacing elements 750 in accordance with another example. Cementless patellofemoral resurfacing system 730 can include one or more slots 702 along at least a portion of ribs 704. One or more slots 702 can be operative to slidingly accommodate one or more ridges 706 attached to at least a portion of an edge of condylar articular surface resurfacing elements 750 so that to firmly lock into place condylar articular surface resurfacing elements 750 once cementless arthroplasty resurfacing system 700 is attached to bone.

As shown in FIG. 7D, the cross section of one or more ridges 706 can be shaped so that attachment portion 712 attached to a border 714 of condylar articular surface resurfacing elements 750 has a smaller thickness than the body of one or more ridges 706 and one or more slots 702 so that one or more ridges 706 can be locked into one or more slots 702 so that to prevent ridge 706 from exiting slot 702 laterally and to allow ridge 706 to slide only along the long axis of one or more slots 702.

Optionally, cementless patellofemoral resurfacing system 730 can also include (a) one or more locking screws 710 to secure condylar articular surface resurfacing elements 750 ridges 706 inside one or more slots 702 by blocking one or more slots 702 thus preventing ridges 706 from sliding out of place unintentionally.

As shown in FIG. 7C, condylar articular surface resurfacing elements 750 can be slid into place along one or more slots 702 as indicated by an arrow designated reference numeral 770 before or after attachment of cementless patellofemoral resurfacing system 730 to bone. Once cementless patellofemoral resurfacing system 730 is attached to bone and condylar articular surface resurfacing elements 750 are in place, the firm attachment of one or more ridges 706 attached to a portion of an edge of condylar articular surface resurfacing elements 750 can be strong enough to obviate one or more attachment tabs 516, requiring less drilling into bone and shortening the length of time required for the arthroplasty procedure.

In the example illustrated in FIG. 7, a cementless arthroplasty system 700 can be a modular system in which cementless patellofemoral resurfacing system 730 serves as a central supporting component for attachable and detachable condylar articular surface resurfacing elements 750 and thus only six tabs 516 can be sufficient to firmly non-breachingly attach both cementless patellofemoral resurfacing system 730 and condylar articular surface resurfacing elements 750 to bone thus providing a non-breaching joint-sparing arthroplastic procedure.

Individual Condylar articular surface resurfacing elements 750 can also be manufactured with 3-D titanium printing technology and can be fully or partially coated with a titanium or titanium alloy micro-granular layer of micro-trabeculae as, for example, Trabecular Structures™ described above to further stimulate bone and/or cartilage growth into micro trabeculae increasing surface contact strength with underlying tissue and limiting its movement after anchoring. Such a micro-granular layer renders cementless arthroplasty resurfacing system 700 not only non-breaching but also a bone/cartilage growth-promoting system.

3-D titanium printing technology can also support precise fitting of cementless individual patellofemoral resurfacing system 730 and condylar articular surface resurfacing elements 750 to the boney surface to be covered.

When attached to bone, one of the points of cementless patellofemoral resurfacing system 730 on which the greatest forces of tension are applied is within the intercondylar fossa 350 (FIG. 3) especially when the knee joint is flexed. Hence, the location and method of attachment of tabs 116/516 in general and especially those within the intercondylar fossa 350 is of major importance.

As disclosed in U.S. Provisional Patent Application No. 62/006186 of the same inventor of the instant application, the stress to which implants are subjected many times impacts the bone screw or pin with which they are attached to bone by bringing about failure of the fixation device. Such failure commonly exhibits itself in the form of loosening, device fatigue and axial pull-out of the device, i.e., axial forces acting, for example on a screw and translated into rotational forces that cause the device to unscrew and loosen bringing about irreversible loss of the bone-implant interface. Since the thread created in the bone cortex by the commonly used screws is relatively shallow, in some cases the bony thread itself may strip and the fixating device can lose its holding power or grip. This is especially true in high tension points such as those affecting cementless patellofemoral resurfacing system 150/730 attachment tabs 116/516 in intercondylar fossa 350.

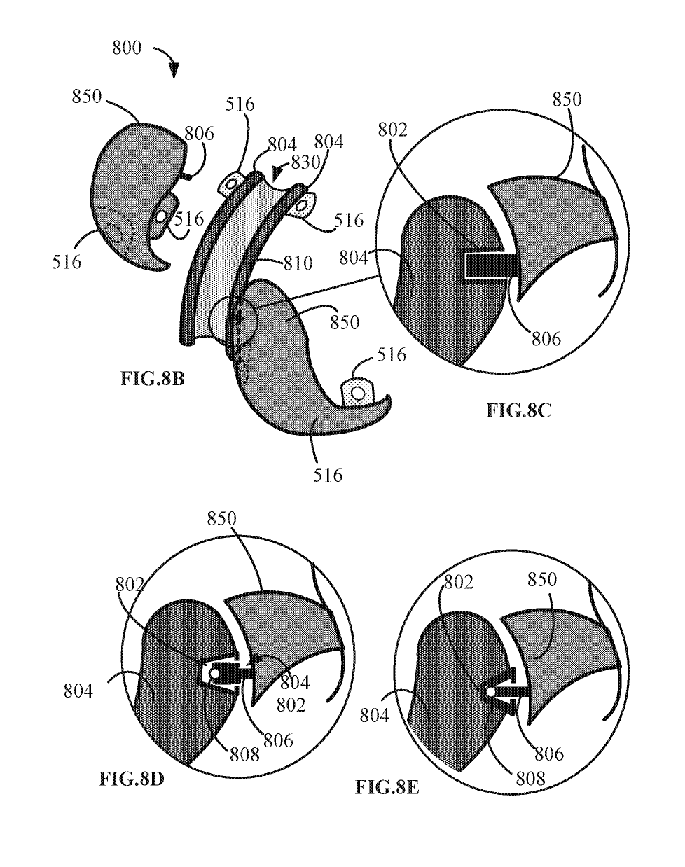

Reference is now made to FIGS. 8A, 8B, 8C, 8D and 8E, together referred to as FIG. 8, which are perspective view and cross-section view simplified illustrations of a cementless arthroplasty resurfacing system 800 in accordance with still another example. Cementless arthroplasty resurfacing system 800 includes cementless patellofemoral resurfacing system 830 and an attachable pair of condylar articular surface resurfacing elements 850. Cementless patellofemoral resurfacing system 830 can include one or more pinholes 802 located in ribs 804 on a side facing away from trough-shaped articular surface 812. One or more pinholes 802 can be operative to slidingly accommodate one or more pins 806 attached to an edge of condylar articular surface resurfacing elements 850 so that to firmly lock into place condylar articular surface resurfacing elements 850 once cementless arthroplasty resurfacing system 800 is attached to bone.

As shown in FIG. 8D, pinhole 802 can be lipped so that to include lips 808 and pin 806 can include an umbrella-spring locking mechanism 810 so that when pin 806 is fully inserted into pinhole 802, umbrella-spring locking mechanism 810 expands as shown in FIG. 8E and is urged against walls of pinhole 802 and lips 808 to lock condylar articular surface resurfacing elements 850 in place.

As shown in FIG. 8, condylar articular surface resurfacing elements 850 can be placed and attached to one or more pinholes 802 before or after attachment of or after attachment of cementless patellofemoral resurfacing system 830 to bone. Once cementless patellofemoral resurfacing system 830 is in place, the firm attachment of one or more pins 806 can be strong enough to obviate one or more attachment tabs 516, requiring less drilling into bone and shortening the length of time required for the arthroplasty procedure.

In the example illustrated in FIG. 8, and similarly to the example of FIG. 8, cementless arthroplasty system 800 can be a modular system in which cementless patellofemoral resurfacing system 830 serves as a central supporting component for attachable and detachable condylar articular surface resurfacing elements 850 and thus only six tabs 516 can be sufficient to firmly non-breachingly attach both cementless patellofemoral resurfacing system 830 and condylar articular surface resurfacing elements 850 to bone thus providing a non-breaching joint-sparing arthroplastic procedure.

When attached to bone, one of the points of cementless patellofemoral resurfacing system 730 on which the greatest forces of tension are applied is within the intercondylar fossa 350 (FIG. 3) especially when the knee joint is flexed. Hence, the location and method of attachment of tabs 116/516 in general and especially those within the intercondylar fossa 350 is of major importance.

The universal implant-to-bone anchoring system structure disclosed in U.S. Provisional Patent Application No. 62/006186, mainly the arrangement of the fins on the anchor shaft is designed so that to stimulate osseous integration in surrounding bone tissue so that to firmly and tightly embed the system anchor in healed bony tissue so that to prevent undesired loosening and axial pull-out of the anchor.

In one example depicted in FIGS. 9A, 9B and 9C, collectively referred to as FIG. 9, which are cross-section and perspective view simplified illustrations of an attachment method of cementless patellofemoral resurfacing system 100/700 to bone, attachment tab 116/516 can be designed to accommodate a universal implant-to-bone fixation system anchor 902 (FIG. 9B, which is section A-A of FIG. 9A) such as that disclosed in U.S. Provisional Patent Application No. 62/006186.

Head 904 of anchor 902 can include a shoulder 906 commonly but not necessarily cylindrical in shape and having one or more walls 908 parallel to shaft 910. Shoulder 906 dimensions can be such so that shoulder 906 can be snugly accommodated in screw hole 118 of cementless patellofemoral resurfacing system 100/700 tab 116/516 when anchor 902 is fully inserted and secured in place, as illustrated in FIG. 9C.

Head 904 can be significantly larger than shaft 910 so that to provide sufficient surface area to urge cementless patellofemoral resurfacing system 100/700 tab 116/516 against the bone when anchor 902 is secured in place in its final position.

Anchor 902 can also include fins 912 arranged on anchor 902 shaft 910 designed so that to stimulate osseous integration in surrounding bone tissue so that to firmly and tightly embed the system anchor in healed bony tissue so that to prevent undesired loosening and axial pull-out of the anchor. The attachment system employing anchor 902 can withstand the extreme forces of tension applied within the intercondylar fossa 350 (FIG. 3).

Failure of attaching any metallic structure to bone can lead to failure that may not necessarily be mechanical in nature. Bringing two dissimilar conducting materials, such as metals, in contact leads to an electrochemical potential difference between them and a development of galvanic corrosion. Aggressive corrosion resulting from an electrical circuit established between the two different metals one of which becomes an anode while the other—a cathode. Common sense would dictate not using multiple metals having a direct contact to each other in an orthopedic implant. Hence, another advantage of employing anchor 902 for the attachment of cementless patellofemoral resurfacing system 100/700 is in that both cementless patellofemoral resurfacing system 100/700 and anchor 902 are made of the same material (e.g., titanium or titanium alloy) and thus do not develop galvanic corrosion.

Reference is now made to FIGS. 10A, 10B and IOC, which are perspective view simplified illustrations of a tibiotalar resurfacing system in accordance with an example. As shown in FIG. 10A tibiotalar resurfacing system 1000 similar in composition to that disclosed in FIG. 1 above, can include a tibial lamellar sheet 1002 having an articular surface 1004 and a bone and/or cartilage (B/C) covering surface 1006. B/C covering surface 1006 can non-breachingly cover healthy or damaged bare bone, cartilage covered bone or partially cartilage covered bone regardless of their condition. Attachment of tibial lamellar sheet 1002 to bone can involve a sawless procedure requiring only a ratchet-type instrument to apply anchors 902 (FIG. 9) through one or more tabs 116 one or more anchor eyes 118 into para-cartilage bone.

Tibiotalar resurfacing system 1000 can also include a tallar lamellar sheet 1008 having an articular surface 1010 and a bone and/or cartilage (B/C) covering surface 1012. B/C covering surface 1012 can non-breachingly cover healthy or damaged bare bone, cartilage covered bone or partially cartilage covered bone regardless of their condition. Attachment of tallar lamellar sheet 1008 to bone can also involve a sawless procedure requiring only a ratchet-type instrument to apply anchors 902 (FIG. 9) through one or more tabs 116 one or more anchor eyes 118 into para-cartilage bone. Both tibial lamellar sheet 1002 bone and/or cartilage (B/C) covering surface 1006 and tallar lamellar sheet 1008 B/C covering surface 1012 can be designed each to wrap around the surface of its respective bone so that to function as a protective cover, a bandage of sorts, protecting damaged underlying bone or cartilage from further erosion.

FIG. 10B illustrated another example of a tibiotalar resurfacing system. Tibiotalar resurfacing system 2000, similar to tibiotalar resurfacing system 1000, also includes a tibial lamellar sheet 2002 having a lip 2006 on one or more sides thereof that is curved so that to cover one or more articular surfaces of the tibia and a tallar lamellar sheet 2004 also having a lip 2008 on one or more sides thereof that is curved so that to cover one or more articular surfaces of the malleolus. Tibial lamellar sheet 2002 and unilateral lip 2006 parallel tallar lamellar sheet 2004 and unilateral lip 2008 parallel each other so that to allow one to glide along the other without any hindrance.

FIG. 10C illustrates an example of implementation of tibiotalar resurfacing system 1000 in the tibiotalar joint.

It will be appreciated by persons skilled in the art that the present method and system are not limited to what has been particularly shown and described hereinabove.

Rather, the scope of the system and devices includes both combinations and sub-combinations of various features described hereinabove as well as modifications and variations thereof which would occur to a person skilled in the art upon reading the foregoing description and which are not in the prior art.

1-58. (canceled)

59. A cementless bony joint resurfacing system, comprising:
at least a first lamellar sheet having an articular surface and a bone covering surface configured to engage an articular surface of a first bone;
at least a second lamellar sheet having an articular surface and a bone covering surface configured to engage an articular surface of a second bone; and
at least one attachment tab coupled to at least one of said lamellar sheets ; and
wherein said system is a joint sparing system, such that when in place:
a) said system is non-breachingly attached to said bones; and
b) said at least one tab is attached to said bone at a joint sparing location.

60. The system according to claim 59, wherein a cross-section geometry of the first articular surface parallels and follows the cross-section geometry of the second articular surface so that said first and second articular surfaces are glidingly moveable along each other.

61. The system according to claim 59, wherein the bone covering surface is at least partially coated with a micro-granular layer of micro-trabeculae to stimulate bone and/or cartilage growth into the micro trabeculae.

62. The system according to claim 59, wherein said first lamellar sheet is a femoral lamellar sheet having a bone covering surface configured to engage a femur articular surface of a user and said second lamellar sheet is a tibial lamellar sheet having a bone covering surface configured to engage a tibia articular surface of a user.

63. The system according to claim 62, wherein the system comprises a patellar lamellar sheet configured to engage a patella articular surface of a user, wherein said articular surface of said first lamellar sheet is configured to receive the articular surface of said patellar lamellar sheet.

64. The system according to claim 62, wherein the one or more ribs protrude from the surface of the at least one of the first and second lamellar sheet and form guard-rail-like structures so that to limit excessive lateral movement (luxation or sub-luxation) of a bone or cartilage gliding over one of the first and/or second lamellar sheet.

65. The system according to claim 62, wherein height of ribs from the articular surface is at least one of constant and variable.

66. The system according to claim 62, wherein at least one of the first and second lamellar sheets also includes a trough or groove along a longitudinal axis (Q-Q).

67. The system according to claim 66, wherein two or more ribs protrude from the surface of one of the first and second lamellar sheets and sandwich said trough or groove therebetween.

68. The system according to claim 66, wherein the first lamellar sheet includes at least one rib along a center of the trough such that the transverse cross section of said trough is W-shaped.

69. The system according to claim 66, wherein the first lamellar sheet includes at least one trough configured to engage at least one rib of said second lamellar sheet, wherein said rib comprises at least one protrusion such that the trough is configured to glide over the rib articular surface.

70. The system according to claim 59, wherein at least one articular surface resurfacing element includes a stopper such that when articulating together with a flat articular surface prevents hyper-extension of a joint.

71. The system according to claim 59, wherein at least one of the articular resurfacing components has a variable thickness.

72. The system according to claim 59, wherein at least one portion of said first lamellar sheet articular resurfacing component is thicker than remaining portions of said first lamellar sheet articular resurfacing component.

73. The system according to claim 59, wherein the system includes one or more slots along at least one edge of a lamellar sheet and/or a rib, such that the slot aligns with at least a portion of a condylar and/or bicondylar articular surface.

74. The system according to claim 73, wherein said slots are configured to receive a locking screw.

75. The system according to claim 59, wherein said first lamellar sheet is a talus lamellar sheet having a bone covering surface configured to engage a talus articular surface of a user and said second lamellar sheet is a tibial lamellar sheet having a bone covering surface configured to engage a distal tibia articular surface of a user.

76. A method for cementless joint resurfacing, comprising:
providing cementless joint resurfacing components including:
at least a first lamellar sheet having an articular surface and a bone covering surface configured to engage an articular surface of a first bone;
at least a second lamellar sheet having an articular surface and a covering surface configured to engage an articular surface of a second bone;
wherein at least one of said lamellar sheets comprises at least one attachment tab; and
locating at least a portion of said at least one tab over at least one para-cartilage area of the bone;
positioning the first lamellar sheet over an articular surface of a first bone;
positioning the second lamellar sheet over an articular surface of a second bone; and
attaching said at least a portion of said at least one tab at said at least one para-cartilage area of the bone, thereby non-breachingly attaching the resurfacing components to a bone and/or cartilage in a joint-sparing manner.

77. The method according to claim 76, wherein attaching the patellofemoral resurfacing components to the bone and/or cartilage employing a sawless procedure.

78. A kit, comprising:
at least one cementless bony joint resurfacing system, comprising:
at least a first lamellar sheet having an articular surface and a bone covering surface configured to engage an articular surface of a first bone;
at least a second lamellar sheet having an articular surface and a covering surface configured to engage an articular surface of a second bone; and
a plurality of universal implant to bone fixation system anchors configured to attach said first and second lamellar sheets to said joint bones, such that when in place,
a) said universal implant to bone fixation system anchors are attached to said bone at a joint sparing location; and
b) said system is non-breachingly attached to said bones.
